(19) United States
(12) Patent Application Publication
(10) Pub. No.: US 2004/0248764 A1
(43) Pub. Date: Dec. 9, 2004

(54) Treatment and prevention of infections in plants

(76) Inventor: Lanny U Franklin, Atlanta, GA (US)

Correspondence Address: NEEDLE & ROSENBERG, P.C., SUITE 1000, 999 PEACHTREE STREET, ATLANTA, GA, 30309-3915, US

(21) Appl. No.: 10/488,130

(22) PCT Filed: Aug. 28, 2002
(86) PCT No.: PCT/US02/27512

Related U.S. Application Data
(60) Provisional application No. 60/315,163, filed on Aug. 28, 2001.
(60) Provisional application No. 60/388,057, filed on Jun. 11, 2002.

(51) Int. Cl. A01N 61/00; A61K 31/00

(52) U.S. Cl. 514/1

(57) ABSTRACT

Composition and methods for prevention and treatment of plant infections. A composition comprising a single terpene, a terpene mixture, or a liposome-terpene(s) composition is disclosed. The composition can be a true solution of an effective amount of an effective terpene and a carrier such as water. The composition can be a suspension or emulsion of terpene, surfactant and carrier (water). The composition(s) of the invention can be administered before or after the onset of the disease. Administration can be, for example, by watering or injecting plants with a solution of the present invention. A true solution of terpene and water can be formed by mixing terpene and water at a solution-forming shear rate in the absence of a surfactant.

### CROSS-REFERENCE TO RELATED APPLICATIONS

This application claims the benefit of U.S. Provisional Application No. 60/315,163, filed Aug. 28, 2001, and U.S. Provisional Application No. 60/388,057, filed Jun. 11, 2002.

### BACKGROUND OF THE INVENTION

1. Field of the Invention

A composition and method for prevention and/or treatment of infections in plants before or after the onset of disease.

2. Background

Plant diseases continue to have significant and identifiable impacts on society, including economic impacts. Plant diseases account for substantial losses in crop yields worldwide and are a great threat to our food supply. Plant disease epidemics have changed the course of history, caused shifts in trading relationships, and changed the face of our landscape. Agriculture is vulnerable to the outbreak of epidemics because of the intensity of crop cultivation and the reliance on a few plant cultivars.

Many Irish descendants in this country are here because potato late blight initiated a food shortage in Ireland in the mid 1800's. As an example of the wider impact of disease, the collapse in Ceylonese coffee bean production began in 1869 due to a coffee rust epidemic and production dropped from 50×10⁶ kg per year to almost nothing by 1890. This outbreak is credited with changing the British from a nation of coffee drinkers to a nation of tea drinkers. The southern corn leaf blight epidemic of 1970 caused a loss of almost all of the maize crop in some states and cost the American economy close to one billion dollars. Soybean stem canker was epidemic in 1983. The citrus industry in Florida was tasked in the early 1900's and again in the 1980's and 1995 by citrus canker and a related disease, both caused by bacteria. The American chestnut tree, for all practical purposes, has been eliminated from this country because of a fungal disease called chestnut blight At the present time, the dreaded Dutch elm disease continues to scourge the country. Pathogen control is, therefore, a vital part of agriculture that is needed to create a stable food supply and to protect populations and economies from the consequences of such pathogen epidemics.

A plant is diseased when its chemistry or structure has submitted to an abnormal, sustained alteration. This definition, although vague, is helpful. The definition indicates that a leaf pulled off a tree is not a disease, but instead an injury, because the alteration is not continuous. Plant diseases are caused by either non-living or living agents. Non-living agents include high or low temperature, atmospheric impurities, mineral deficiencies, mineral excesses, or possibly other causes. The living agents that cause plant diseases include fungi, bacteria, a few higher plants, nematodes, algae, viruses, mycoplasmas, and viroids. A fungus, bacterium, or virus, for example, enters a plant and continues to deprive the plant of nourishment or continuously alters normal functions of the plant

Disease by an infective agent impairs necessary functions of the plants. Some diseases block water-conducting vessels in the plant which results in a wilted condition similar to drought. Root rots destroy the roots that absorb water and nutrients from the soil. Leaf spotting diseases reduce photosynthesis in the plant which results in less food manufactured by the plant. Seeds, seed pieces, fruits, and flowers may be destroyed by rots or blights. Diseases of this type reduce the reproductive ability of a plant, and in the case of ornamentals, the disease is unsightly.

A susceptible plant, an agent causing the disease, and a suitable environment are all necessary components for disease to occur. For example, fungi that cause leaf spots need a susceptible host, moist conditions on the leaves, and favorable temperatures so that spores will germinate. Many root rotting fungi need a susceptible host coupled with high soil moisture or a soil pH favorable for fungus growth

Fungi

Fungi are plants that lack chlorophyll, stems, leaves, and roots. Their vegetative body is made up of microscopic, tubular structures called hyphae, amoeboid structures called plasmodia, or single budded cells. (Some newer classification schemes do not include all fungi with hyphae or fungi with amoeboid structures as “true” fungi.) Fungi grow on or in the soil or within or on host tissue. Fungi are further characterized by the production of microscopic “seeds” called spores. Fungi produce different types of spores. Spores may be spread by wind, insects, rain, or irrigation water. Some spores are suitable for wind or water dissemination while others have thick walls, thereby being adapted for survival in soil or other concealed places for many years. Some spores serve as carriers of new genetic traits.

Fungi also spread when infected plants (including seed) are moved from one location to another. Similarly, fingi may be carried on a tractor or maintenance implements, or people working within the planting, or livestock. Fungi can infect plant parts when wounds are made by harvesting, farm implements, hail, wind, blowing sand, insects, nematodes, or other fungi.

Many fungi can live as saprophytes in the soil or decaying plant litter as well as being parasitic. Fungi that can grow saprophytically on old crop debris and soil usually can be grown as a culture on a growth medium in the laboratory. Some fungi, however, such as rusts, downy mildews, and powdery mildews, are obligate parasites, i.e., they normally grow only in a living plant. Certain rusts have been cultured in a laboratory.

Viruses

Most viruses are particles made up of a nucleic acid core (RNA or DNA) and a protein coat. No cellular structure is present, although some viruses may be enclosed by a membrane. Viruses are obligate parasites which reproduce in living cells of susceptible host plants. Virus particles are not visible with light microscopes; an electron microscope is used to reveal their structure.

Viruses are spread by mechanical rubbing of one infected plant on another, insects, fungi, nematodes, transporting of infected plants from one location to another, seeds, seed pieces, grafting, dodder, farm equipment, and man's hands. Viruses can enter a plant through wounds. When an insect or nematode feeds on a plant, the virus passes from the insect into the plant or the insect acquires the virus from the plant. Fungi are vectors for certain viruses.

Viroids

Viroids are low molecular weight nucleic acids that have been associated with certain plant diseases. Viroids are similar to a virus, but lack protein encapsulation. Viroids causing plant diseases contain RNA only and, therefore, are the smallest known infectious agents causing plant diseases. Viroids are spread by implements or other mechanical devices.

Algae

Algae resemble fungi in size and structure but differ primarily by the presence of chlorophyll in algae and the absence of chlorophyll in fungi. Algae have unicellular, colonial, and filamentous species. A few are parasitic in plants grown in subtropical or tropical environments.

Bacteria/Mollicutes

Bacteria are microscopic, one-celled organisms which increase by division of cells. Some bacteria, under favorable conditions, can divide every 20 minutes. In 24 hours the division could result in 300 billion new individuals. Bacteria can be grown as cultures in a laboratory. Bacteria survive on or in host plants, susceptible weeds, and organic debris in soil.

Bacteria are spread by insects, irrigation water, rain, movement of infected plants, seeds, seed pieces, grafting, livestock, and farm equipment. Bacteria enter plants through wounds or natural plant openings such as stomata, lenticels, or hydathodes. When plant tissue is gorged with water, bacterial ingress into plant tissue increases.

Mollicutes is a class of cell wall-less prokaryotes that are the smallest, simplest, self-replicating prokaryotes. Evolutionarily, mollicutes are closely similar to their bacterial counterparts. Mollicutes includes phytoplasmas, mycoplasmas, spiroplasmas, Acheolplasmas, and entomoplasmas (Razin et al., 1998, Molecular biology and pathogenicity of mycoplasmas, Micro. Mol. Bio. Rev. 62:1094-1156). The mollicutes associated with plants are phloem-restricted pathogens (spiroplasmas, mycoplasma-like organisms) or surface contaminants (Spiroplasma spp., Mycoplasma spp., Acholeplasma spp., and others).

The plant pathogenic mollicutes are transmitted by insect vectors. Mycoplasma are dispersed by leafhoppers or moving infected plants. Many other insects carry mollicutes, particularly spiroplasmas, and deposit these organisms on plant surfaces where other insects pick them up. New acholeplasma, mycoplasma, and spiroplasma species have been identified in insect hosts or on plant surfaces.

Mycoplasma are small parasitic organisms that have long been known to cause disease in plants. The organisms produce spherical- to ellipsoid-shaped bodies that are smaller than bacteria, but larger than most virus particles. Mycoplasma live in phloem of cells of plants. Mycoplasma contain protein, DNA, RNA, and enzymes. The mycoplasmas' elementary bodies vary in shape and size. Many plant diseases, previously thought to be caused by viruses, are now known to be caused by mycoplasmas. Mycoplasma are sensitive to heat and some antibiotics.

The “gold standard” for detection of mycoplasma genomes is the polymerase chain reaction (PCR), however, confirmation of PCR is often done by southern blot and molecular probes.

Like mycoplasmas, phytoplasmas are organ/tissue specific to an extent. Phytoplasmas are extremely small, phloem-limited plant pathogenic bacteria-like prokaryotes that lack a cell wall. Phytoplasmas like roots very well, but can be found in many places in the plant (see, e.g., Siddique et al., 1998, Histopathology and within-plant distribution of the phytoplasma associated with Australian papaya dieback, Plant dis. 82(10):1112-1120). Many plant diseases once thought to be caused by viruses are now known to be caused by phytoplasmas. Phytoplasmas are tansmitted by grafting, dodder, and insects. Phytoplasmas are known to be transmitted by over 100 species of insects, including leaf hoppers (a primary vector), planthoppers, and psyllids. Phytoplasmas might also be seed-borne.

Unlike typical bacteria, phytoplasmas cannot be cultured on artificial media in the laboratory. Phytoplasmas must be maintained in the host. Maintenance of phytoplasmas can be done in plant tissue culture, continuous graft or insect transmission, or freezing leafhoppers (Bertaccini et al., 1992, Lee and Chiykowski, 1963 Infectivity of aster yellows virus preparations after differential centrifugations of extract from viruliferous leafhoppers, Virol. 21:667-669). Phytoplasmas can be detected with phytoplasma-specific stains such as the 4,6-diamidino-2-pheylindole (DAPI) (Sinclair, W. A., R. J. Iuli, A. T. Dyer, and A. O. Larsen, 1989, Sampling and histological procedures for diagnosis of ash yellows. Plant disease. 73:432435) and Dienes' stain (Deeley et al., 1979, Use of Dienes' Stain to detect plant diseases induced by MLOs. Phytopathology. 69:1169-1171). Phytoplasmas can also be detected using electron microscopy and molecular techniques including DNA probes, polymerase chain reaction (PCR), and enzyme linked immuno-absorbent assay (ELISA). Example articles demonstrating these types of techniques include Gunderson and Lee, 1996, Ultrasensitive detection of phytoplasmas by nested-PCR assays using two universal primer pairs. Phytopath. Medit. 35:144-151; Gunderson et al., 1996, Genomic diversity and differentiation among phytoplasma strains in 16S rRNA groups I (aster yellows and related phytoplasmas) and M (X-disease and related phytoplasmas). International J. of Syst. Bact. 46(1):64-75; Lee et al., 1991, Genetic Interrelatedness among clover proliferation mycoplasmalike organisms (MLOs) and other MLOs investigated by nucleic acid hybridization and restriction fragment length polymorphism analyses. Appl. Environ. Micro. 57(12):3565-3569; Lee et al., 1993, Universal amplification and analysis of pathogen 16S rDNA for classification and identification of mycoplasmalike organisms. Phytopathology. 83:834-842; Schaff et al., 1992, Sensitive detection and identification of mycoplasma-like organisms in plants by polymerase chain reactions Biochem. Biophys. Res. Comm. 186:1503-1509; and Lee et al., 1998, Revised classification scheme of phytoplasmas based on RFLP analyses of 16S rRNA and ribosomal protein gene sequence [Review]. International Journal of Systematic Bacteriology. 48: 1153-1169). A review of how to handle phytoplasmas can be found in S. J. Eden-Green (1982) Culture of other microorganisms from yellows-diseased plants, pp.201-239. In M. J.D.a.P.G. Markham (ed.), Plant and Insect mycoplasma techniques. Croom and Helm, London.

Review articles and books on phytoplasmas (McCoy et al., 1989) and mycoplasmas (Razin et al., 1998) can be found. Others include Markham, 1982, The “yellows” plant diseases: plant hosts and their interaction with the pathogens, pp. 82-100 In M. J. Daniels and P. G. Markham (Eds.) Plant and Insect mycoplasma techniques Croom Helm, London and Kirkpatrick, 1989, Strategies for characterizing plant pathogenic MLO and their effects on plants, pp. 241-293. In T. Kosuge and E. W. Nester (eds.), Plant-Microbe interactions: molecular and genetic perspectives, vol. 3, McGraw-Hill, NY; and Smart 1995.

Spiroplasma species are also a member of Mollicutes. A number of assays are available for the detection and characterization of the culturable plant pathogenic spiroplasmas, unlike the non-culturable mycoplasma-like organisms (MLO).

Diseases

The above-described infective agents cause diseases in a variety of plants. Many of these plants are economically significant crops. Examples of these economically significant plants include grapes, stone fruits, and coffee.

One bacterium responsible for plant infections is Xylella, such as Xylella fastidiosa. Xylella fastidiosa is a gram-negative, xylem-limited bacterium capable of affecting economically important crops. The bacterium has a large host range, including at least 28 families of both monocotyleyledonous and dictotyledonous plants. Plant hosts for X. fastidiosa include miscellaneous ornamentals, grape, oleander, oak, almond, peach, pear, citrus, coffee, maple, mulberry, elm, sycamore, and alfalfa, where the bacterium inhabits the plants' xylem. Other strains of Xylella cause important diseases of peach, citrus, coffee, and numerous forest tree species. Vectors, such as insects like xylem sap-feeding leafhoppers, acquire the bacterium by feeding on infected plants and subsequently infect other plants. Xylella can also be graft transmitted.

Pierce's Disease (PD), a lethal disease of grapevine, is caused by the bacterium Xylella fastidiosa and is spread by certain kinds of leafhoppers known as sharpshooters. The bacterium is limited to the grapevine xylem. Insects with piercing/sucking mouthparts that feed on xylem sap transmit the bacteria from diseased to healthy plants. Vines develop symptoms when the bacteria block the water conducting system and reduce the flow of water to affected leaves. Water stress begins in mid-summer and increases through fall. The first evidence of PD infection usually is a drying or “scorching” of leaves. About mid-growing season, when foliar scorching begins, some or all of the fruit clusters may wilt and dry up. The bark on affected canes often matures unevenly, leaving islands of mature (brown) bark surrounded by immature (green) bark or the reverse. Chronically affected vines are slow to begin growth, becoming somewhat dwarfed or stunted and some canes or spurs may fail to bud at all. A vine infected with Pierce's Disease usually becomes non-productive and dies within two years and produces no crop.

Pierce's Disease is known from North America through Central America and has been reported in some parts of northwestern South America. It is present in some California vineyards every year, with the most dramatic losses occurring in the Napa Valley and in parts of the San Joaquin Valley. PD has cost the California wine and grape industries millions of dollars in lost revenues since it began destroying grapevines in Napa and Sonoma counties. Economic damages from the disease have been estimated to cost as much as $20,000 per acre. During severe epidemics, losses to PD may require major replanting. Currently there are more than 500 million commercial grapevines in the United States, with 40% of the acreage at risk for significant economic loss. The recent outbreak of Pierce's Disease in California has also had a major impact on the state's nursery business due to quarantines imposed in efforts to prevent the spread of the disease. In Florida and other southeastern states, the disease is considered to be the single most formidable obstacle to the growing of European grape varieties. This has precluded commercial production of European varieties (some muscadine grapes and hybrids of American wild grape species with European grapes (Vitis vinifera) are tolerant or resistant to PD).

Since the mid-1970s, other strains of Xylella fastidiosa have been discovered, and almost all of these cause leaf scorching of woody perennials, such as American elm, maple, mulberry, or plum. In some plants, such as peach and alfalfa, the bacterium slows and stunts plant growth. Xylella sp. are responsible for variegated chlorosis in citrus, almond leaf scorch disease, phony peach disease, alfalfa dwarf, and others. Xylella fastidiosa attacks citrus fruits by blocking the xylem, resulting in juiceless fruits of no commercial value.

Infection Control

Modern pathogen control is multidisciplinary, and relies on techniques such as the use of vector management, crop rotation, the production of pathogen-free plant seeds, and chemical control measures. It has been estimated that despite these controls, 10 to 15% of world food production is lost to pathogens and the effects of pathogens.

Diseases caused by non-living agents are often controlled by simply adding fertilizer, avoiding excess amounts of fertilizer, controlling an air pollution source, or protecting plants from the adversities of the weather. Plants affected by non-living agents are often more susceptible to living infective agents.

Diseases caused by living infective agents are controlled by various methods including eradication, plant surgery, proper sanitation, crop rotation, control of vectors, and chemicals. Development of resistant varieties is often considered to be the best means of control.

Eradication and exclusion have been effective for controlling several diseases. Exclusion of disease is one of the purposes of quarantines. Eradication of disease may be done by other means also, for example, by removal of other species of plants that are also hosts of the disease. These plants may be weeds or alternate hosts. Alternate hosts support part of the life cycle of the organism causing disease. Destroying diseased plants in a crop can be used in controlling plant disease.

Surgery of plants can be used to control plant diseases. For example, a bacterial disease of woody plants called fire blight can be reduced by removing and destroying infected branches.

Sanitation around propagating beds, greenhouses, and fields is a known control measure.

Crop rotation is another method whereby disease can be reduced. Crop rotation is done by alternating a given crop with non-susceptible crops. Crop rotation is less effective for controlling obligate parasites that produce wind blown spores.

The selection of disease-free stock is another known control measure. Use of disease-free seed follows this same principle of control.

Proper use of fertilizer can reduce disease. Some diseases are suppressed by reduced amounts of nitrogen; others are suppressed by increased amounts of nitrogen. Increased amounts of calcium in plant tissue often suppress disease. Proper ratios of certain elements in fertilizers can be used to suppress plant diseases.

Control of insects and nematodes often reduce disease when a disease-causing organism is partly or wholly dependent upon these organisms. Insects and nematodes not only act as vectors, but also their damage can provide an entrance point for disease-causing organisms.

Weed control is beneficial for disease control; weeds can harbor inoculum, interfere with spray deposition, reduce plant vigor, and reduce aeration within crop canopies.

Finally, timely applications of chemicals are used to control many plant diseases. Where a pathogen is consistently challenged by the same pesticide over time, individuals within the population that are resistant to the pesticide gradually predominate.

Farmers and agribusiness are heavily reliant on using chemical control measures to combat pathogens or pathogen vectors, as well as on the breeding of new, pathogen-resistant plant lines. These approaches have considerable disadvantages and often fail to protect crops. Chemical controls, like pesticides and fungicides, are expensive and environmentally undesirable. Breeding new plant lines is an expensive long term process.

Methods of treating or preventing infection by Xylella which have been tried include control of the insect vectors (such as through pesticide use or physical barriers), destruction of infected plants, and pruning and freezing.

Scientists are evaluating other methods, including the use of other bacterial species and bacteriophages, for the control of Xylella fastidiosa in host plants. Prevention methods against PD include the use of broad-spectrum antibiotics or boosting levels of essential plant bacterial micronutrients such as zinc, iron, copper, and molybdenum that could be toxic to Xylella sp. Another way to prevent the infection is by genetically modifying the chemistry and structure of the xylem making it uninhabitable for the bacteria, such as shown in U.S. Pat. No. 6,232,528. The patent covers introduction and expression in grape of a gene that produces a polypeptide from a wild silk moth for lytic peptides that kills bacteria, including the Pierce's Disease bacterium.

Mycoplasma causes disease such as X-disease in orchard trees, e.g., peaches, nectarines, and cherries. Symptoms are primarily foliar, but fruits may also be affected. Disease is transmitted by vectors such as leafhoppers. There is no chemical means for protecting trees from X-disease. Leafhopper control may reduce the spread of disease. Identifying and eradicating inoculun sources have been the better choice for prevention.

Prior methods of “curing” a plant of phytoplasmas include heat treatment and/or by passing them through tissue culture (Kunkel, 1941, Heat cure of aster yellows in periwinkles, Am. J. Botany 28:761-769). This is a very difficult process, and it is easier to pass the phytoplasma-infected plant through a seed cycle, since phytoplasmas are not seed transmitted. Remission of symptoms and even curing a plant can be achieved through the application of the antibiotic tetracycline (McCoy and Williams, 1982, Chemical treatment for control of plant mycoplasma diseases, pp. 152-173, In M. J. Daniels and D. S. Williams (eds.), Plant Insect Mycoplasma Techniques. London, Croom Helm).

Injections of antibiotics can be used to treat diseased plants, but the treatment procedure is labor-intensive, must be done during specific times of the year, and must be repeated annually to prevent a relapse. Most growers consider it more cost-effective to remove diseased plants and replant in their place.

It is also known that overuse of antibiotics induces resistance in bacteria. This is true not only in treatment of humans but also the prophylactic and treatment uses of antibiotics in agriculture. Computer models show that heavy agricultural use of antibiotics dramatically increases the rate at which new resistant strains of bacteria move into human populations. For example, the use of quinolones, a class of antibiotics widely used in feedlots and in human use, has caused the spread of resistant strains of Campylobacter jejuni. Until recently, quinolones were almost always effective in treating severe cases of illness, but new studies have shown that I in 5 human Campylobacter infections is resistant to most quinolones, as is a significant portion of the same bacteria found in chicken. The National Foundation for Infectious Diseases estimates that antibiotic resistance costs the U.S. $4 billion a year, and some highly resistant strains of infectious bacteria are all but untreatable now. Toner; M., “Report: Farms raising germs' resistance,” Atlanta Journal Constitution, Apr. 23, 2002,p. A-7.

For the above reasons, and others, it is desirable to find additional methods for controlling plant infections that are environmentally-friendly, acceptable to consumers, and avoid other drawbacks of previous methods.

### SUMMARY OF THE INVENTION

In accordance with the purpose(s) of this invention, as embodied and broadly described herein, this invention relates to prevention and/or treatment of plant infections.

The present invention provides compositions and methods for treating and/or preventing plant infections that avoid drawbacks found in the previous methods.

The present invention provides a composition for treating and/or preventing infections in plants comprising an effective amount of at least one effective terpene. The composition can be a solution capable of being taken up by a plant, a true solution. The composition can further comprise water. The composition can further comprise a surfactant and water.

In the composition of the invention which contains a surfactant, the surfactant an be, for example, polysorbate 20, polysorbate 80, polysorbate 40, polysorbate 60, olyglyceryl ester, polyglyceryl monooleate, decaglyceryl monocaprylate, propylene glycol dicaprilate, triglycerol monostearate, TWEEN, SPAN 20, SPAN 40, SPAN 60, SPAN 80, or mixtures thereof. The composition can comprise about 1 to 99% by volume terpenes and about 1 to 99% by volume surfactant.

The composition of the invention can comprise a mixture of different terpenes or a terpene-liposome (or other vehicle) combination.

The terpene of the composition can comprise, for example, citral, pinene, nerol, b-ionone, geraniol, carvacrol, eugenol, carvone, terpeniol, anethole, camphor, menthol, limonene, nerolidol, farnesol, phytol, carotene (vitamin A₁), squalene, thymol, tocotrienol, perillyl alcohol, borneol, myrcene, simene, carene, terpenene, linalool, or mixtures thereof.

The composition can comprise between about 20 ppm and about 5000 ppm of the terpene, specifically about 125, 250, or 500 ppm.

The composition is effective against various infective agents including bacteria, mycoplasmas/phytoplasmas, and/or fingi.

A composition for treating and/or preventing infections in plants comprising a true solution comprising an effective amount of at least one effective terpene and water is disclosed.

A method for preventing and/or treating plant infection comprising administering a composition comprising an effective amount of an effective terpene to plants is also disclosed. The administration of the method can be by spraying or watering the plants with the composition or by injecting plants with the composition, for example. The injection can be into the xylem of the plant.

The methods are practiced using the compositions of the present invention.

The plants can be, for example, grape vines, stone fruit trees, coffee, or ornamental plants, especially grape vines.

The composition can be made by mixing an effective amount of an effective terpene and water. The mixing can be done at a solution-forming shear until formation f a true solution of the terpene and water; the solution-forming shear can be by high shear or high pressure blending or agitation.

A method of the present invention for preventing and/or treating plant infections comprises administering a composition comprising an effective amount of an effective terpene and water to plants, such as a true solution of the terpene and water. The invention includes a method for making a terpene-containing composition effective for preventing and/or treating plant infections comprising mixing a composition comprising a terpene and water at a solution-forming shear until a true solution of the terpene is formed.

The invention further includes a method for making a terpene-containing composition capable of plant root uptake and effective for preventing and/or treating plant infections comprising adding terpene to water, and mixing the terpene and water under solution-forming shear conditions until a true solution of terpene and water forms.

A composition of the present invention comprises an effective amount of an effective terpene.

The composition can be a true solution of terpene and water.

Terpenes are widespread in nature. Their building block is the hydrocarbon isoprene (C₅H₈). Examples of terpenes include citral, pinene, nerol, b-ionone, geraniol, carvacrol eugenol, carvone, terpeniol, anethole, camphor, menthol, limonene, nerolidol, farnesol, phytol, carotene (vitamin A₁), squalene, thymol, tocotrienol, perillyl alcohol, borneol, myrcene, simene, carene, terpenene, and linalool.

Terpenes have previously been found to inhibit the in vitro growth of bacteria and some external parasites. Geraniol was found to inhibit growth of two fungal strains. B-ionone has antifungal activity which was determined by inhibition of spore germination and growth inhibition in agar. Teprenone (geranylgeranylacetone) has an antibacterial effect on H. pylori. Solutions of 11 different terpenes were effective in inhibiting the growth of pathogenic bacteria (five food borne pathogens) in in vitro tests; levels ranging between 100 ppm and 1000 ppm were effective. The terpenes were diluted in water with 1% polysorbate 20. Diterpenes, i.e., trichorabdal A (from R. Trichocarpa) has shown a very strong antibacterial effect against H. pylori.

The present invention includes methods of making the compositions and methods of using the compositions.

A method of making the composition comprises adding a terpene to a carrier.

A method of treating and/or preventing plant infections comprises administering a composition comprising a terpene and a carrier to a plant.

Additional aspects and advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate results of the invention and together with the description, serve to explain the principles of the invention.

FIG. 1 shows an untreated grapevine infected with Xylella.

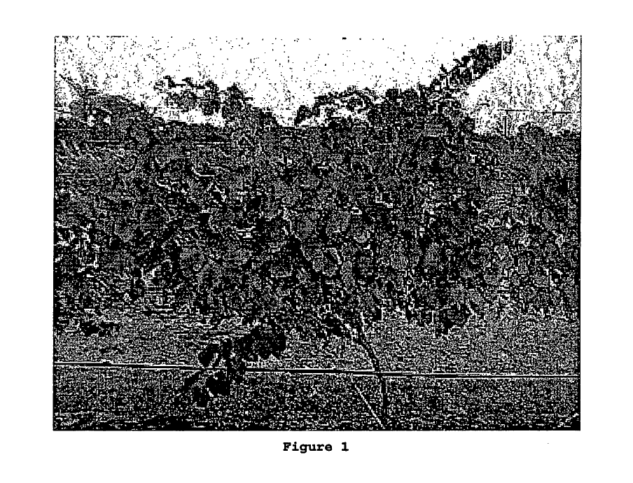

FIG. 2 shows an untreated grapevine infected with Xylella.

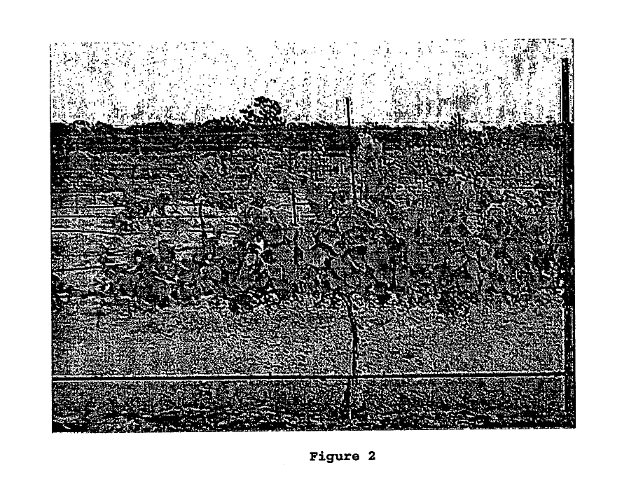

FIG. 3 shows a grapevine infected with Xylella which was treated once with the composition of the present invention over 7 months prior to the photograph.

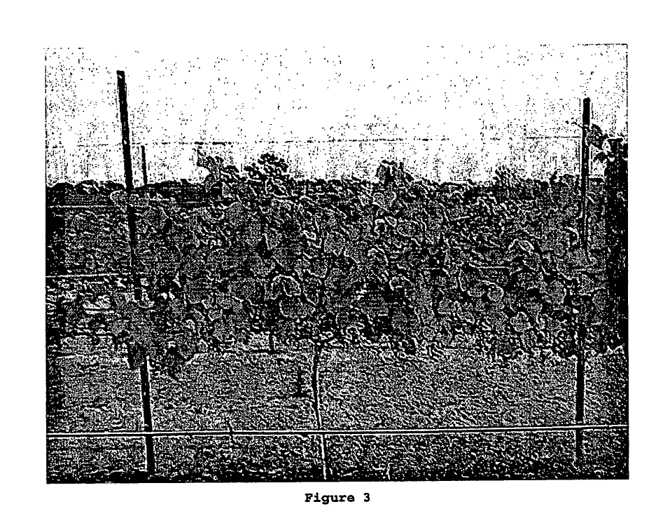

FIG. 4 shows a grapevine infected with Xylella which was treated once with the composition of the present invention over 7 months prior to the photograph.

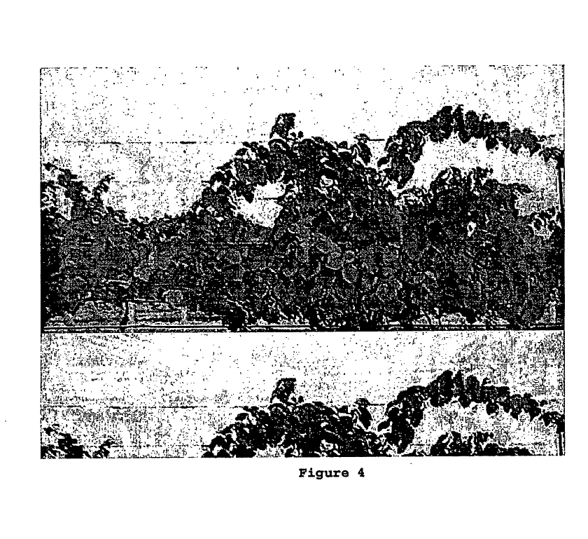

### DETAILED DESCRIPTION OF THE INVENTION

Definitions

Before the present compounds, compositions, articles, devices, and/or methods are disclosed and described, it is to be understood that this invention is not limited to specific synthetic methods, specific methods of making the terpenes or compositions as such may, of course, vary. It is also to be understood that the terminology used herein is for the purpose of describing particular embodiments only and is not intended to be limiting.

It must be noted that, as used in the specification and the appended claims, the singular forms “a,” “an,” and “the” include plural referents unless the context clearly dictates otherwise. Thus, for example, reference to “a terpene” includes mixtures of terpenes, reference to “a carrier” includes mixtures of two or more carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

A volume percent of a component, unless specifically stated to the contrary, is based on the total volume of the formulation or composition in which the component is included.

“Optional” or “optionally” means that the subsequently described event or circumstance may or may not occur, and that the description includes instances where said event or circumstance occurs and instances where it does not. For example, the phrase “optionally surfactant” means that the surfactant may or may not be added and that the description includes both with a surfactant and without a surfactant where there is a choice.

By the term “effective amount” of a compound or property as provided herein is meant such amount as is capable of performing the function of the compound or property for which an effective amount is expressed, such as a non-phytotoxic but sufficient amount of the compound to provide the desired function, i.e., anti-infective. As will be pointed out below, the exact amount required will vary from subject to subject (plant to plant, field to field), depending on the subject, and general condition of the subject, the severity of the disease that is being treated, the particular compound used, its mode of administration, and the like. Thus, it is not possible to specify an exact “effective amount.” However, an appropriate effective amount may be determined by one of ordinary skill in the art using only routine experimentation.

By the term “effective terpene” is meant a terpene which is effective against the particular infective agent of interest.

By the term “true solution” is meant a solution (essentially homogeneous mixture of a solute and a solvent) in contrast to an emulsion or suspension. A visual test for determination of a true solution is a clear resulting liquid. If the mixture remains cloudy, or otherwise not clear, it is assumed that the mixture formed is not a true solution but instead a mixture such as an emulsion or suspension.

Composition(s)

The compositions of the present invention comprise isoprenoids. More specifically, the compositions of the present invention comprise terpenoids. Even more specifically, the compositions of the present invention comprise terpenes. Terpenes are widespread in nature, mainly in plants as constituents of essential oils. Terpenes are unsaturated aliphatic cyclic hydrocarbons. Their building block is the hydrocarbon isoprene (C₅H₈)ₙ. A terpene is any of various unsaturated hydrocarbons, such as C₁₀H₁₆, found in essential oils, oleoresins, and balsams of plants, such as conifers. Some terpenes are alcohols (e.g., menthol from peppermint oil), aldehydes (e.g., citronellal), or ketones.

Terpenes have been found to be effective and nontoxic dietary antitumor agents, which act through a variety of mechanisms of action. Crowell, P. L. and M. N. Gould, 1994. Chemoprevention and Therapy of Cancer by D-limonene, Crit. Rev. Oncog. 5(1): 1-22; Crowell, P. L., S. Ayoubi and Y. D. Burke, 1996, Antitumorigenic Effects of Limonene and Perillyl Alcohol Against Pancreatic and Breast Cancer, Adv. Exp. Med. Biol. 401: 131-136. Terpenes, i.e., geraniol, tocotrienol, perillyl alcohol, b-ionone, and d-limonene, suppress hepatic HMG-COA reductase activity, a rate limiting step in cholesterol synthesis, and modestly lower cholesterol levels in animals. Elson C. E. and S. G. Yu, 1994, The Chemoprevention of Cancer by Mevalonate-Derived Constituents of Fruits and Vegetables, J. Nutr. 124: 607-614. D-limonene and geraniol reduced mammary tumors (Elgebede, J. A., C. E. Elson, A. Qureshi, Mass. Tanner and M. N. Gould, 1984, Inhibition of DMBA-Induced Mammary Cancer by Monoterpene D-limonene, Carcinogensis 5(5): 661-664; Elgebede, J. A., C. E. Elson, A. Qureshi, M. A. Tanner and M. N. Gould, 1986, Regression of Rat Primary Mammary Tumors Following Dietary D-limonene, J. Nat'l Cancer Institute 76(2): 323-325; Karlson, J., A. K. Borg, R. Unelius, M. C. Shoshan, N. Wilking, U. Ringborg and S. Linder, 1996, Inhibition of Tumor Cell Growth By Monoterpenes In Vitro: Evidence of a Ras-Independent Mechanism of Action, Anticancer Drugs 7(4): 422-429) and suppressed the growth of transplanted tumors (Yu, S. G., P. J. Anderson and C. E. Elson, 1995, The Efficacy of B-ionone in the Chemoprevention of Rat Mammary Carcinogensis, J. Angri. Food Chem. 43: 2144-2147).

Terpenes have also been found to inhibit the in vitro growth of bacteria and fungi (Chaumont J. P. and D. Leger, 1992, Campaign Against Allergic Moulds in Dwellings, Inhibitor Properties of Essential Oil Geranium “Bourbon, “Citronellol, Geraniol and Citral, Ann. Pharm. Fr. 50(3): 156-166), and some internal and external parasites (Hooser, S. B., V. R. Beasly and J. J. Everitt, 1986, Effects of an Insecticidal Dip Containing D-limonene in the Cat, J. Am. Vet. Med. Assoc. 189(8): 905-908). Geraniol was found to inhibit growth of Candida albicansand Saccharomyces cerevisiae strains by enhancing the rate of potassium leakage and disrupting membrane fluidity (Bard, M., M. R. Albert, N. Gupta, C. J. Guuynn and W. Stillwell, 1988, Geraniol Interferes with Membrane Functions in Strains of Candida and Saccharomyces, Lipids 23(6): 534-538). B-ionone has antifungal activity which was determined by inhibition of spore germination, and growth inhibition in agar (Mikhlin E. D., V. P. Radina, A. A. Dmitrossky, L. P. Blinkova, and L. G. Button, 1983, Antifungal and Antimicrobial Activity of Some Derivatives of Beta-Ionone and Vitamin A, Prild Biokhim Mikrobiol, 19: 795-803; Salt, S. D., S. Tuzun and J. Kuc, 1986, Effects of B-ionone and Abscisic Acid on the Growth of Tobacco and Resistance to Blue Mold, Mimicry the Effects of Stem Infection by Peronospora Tabacina, Adam Physiol. Molec. Plant Path 28:287-297). Teprenone (geranylgeranylacetone) has an antibacterial effect on H. pylori (Ishii, E., 1993, Antibacterial Activity of Terprenone, a Non Water-Soluble Antiulcer Agent, Against Helicobacter Pylori, Int. 3. Med. Microbiol. Virol. Parasitol. Infect Dis. 280(1-2): 239-243). Solutions of 11 different terpenes were effective in inhibiting the growth of pathogenic bacteria in in vitro tests; levels ranging between 100 ppm and 1000 ppm were effective. The terpenes were diluted in water with 1% polysorbate 20 (Kim, J., M. Marshall and C. Wei, 1995, Antibacterial Activity of Some Essential Oil Components Against Five Foodborne Pathogens, J. Agric. Food Chem. 43: 2839-2845). Diterpenes, i.e., trichorabdal A (from R. Trichocarpa) has shown a very strong antibacterial effect against H. pylori (Kadota, S., P. Basnet, E. Ishii, T. Tamura and T. Namba, 1997, Antibacterial Activity of Trichorabdal A from Rabdosia Trichocarpa Against Helicobacter Pylori, Zentralbl. Bakteriol 287(1): 63-67).

Rosanol, a commercial product with 1% rose oil, has been shown to inhibit the growth of several bacteria (Pseudomonas, Staphylococus, E. coli, and H. pylori). Geraniol is the active component (75%/o) of rose oil. Rose oil and geraniol at a concentration of 2 mg/L inhibited the growth of H. pylori in vitro. Some extracts from herbal medicines have been shown to have an inhibitory effect in H. pylori, the most effective being decursinol angelate, decursin, magnolol, berberine, cinnamic acid, decursinol, and gallic acid (Bae, E. A., M. J. Han, N. J. Kim, and D. H. Kim, 1998, Anti-Helicobacter Pylori Activity of Herbal Medicines, Biol. Pharm. Bull. 21(9) 990-992). Extracts from cashew apple, anacardic acid, and (E)-2-hexenal have shown bactericidal effect against H. pylori. There may be different modes of action of terpenes against microorganism; they could (1) interfere with the phospholipid bilayer of the cell membrane, (2) impair a variety of enzyme systems (HMG-reductase), and (3) destroy or inactivate genetic material.

It is believed that due to the modes of action of terpenes being so basic, e.g., blocking of cholesterol, that infective agents will not be able to build a resistance to terpenes.

Terpenes, which are Generally Recognized as Safe (GRAS), have been found to inhibit the growth of cancerous cells, decrease tumor size, decrease cholesterol levels, and have a biocidal effect on microorganisms in vitro. Owawunmi, G. O., 1989, Evaluation of the Antimicrobial Activity of Citral, Letters in Applied Microbiology 9(3): 105-108, showed that growth media with more than 0.01% citral reduced the concentration of E. coli, and at 0.08% there was a bactericidal effect. Barranx, A. M. Barsacq, G. Dufau, and J. P. Lauilhe, 1998, Disinfectant or Antiseptic Composition Comprising at Least One Terpene Alcohol and at Lease One Bactericidal Acidic Surfactant, and Use of Such a Mixture, U.S. Pat. No. 5,673,468, teach a terpene formulation, based on pine oil, used as a disinfectant or antiseptic cleaner. Koga, J. T. Yamauchi, M. Shimura, Y. Ogasawara, N. Ogasawara and J. Suzuki, 1998, Antifungal Terpene Compounds and Process for Producing the Same, U.S. Pat. No. 5,849,956, teach that a terpene found in rice has antifungal activity. Iyer, L. M., J. R. Scott, and D. F. Whitfield, 1999, Antimicrobial Compositions, U.S. Pat. No. 5,939,050, teach an oral hygiene antimicrobial product with a combination of 2 or 3 terpenes that showed a synergistic effect. Several U.S. patents (U.S. Pat. Nos. 5,547,677, 5,549,901, 5,618,840, 5,629,021, 5,662,957, 5,700,679, 5,730,989) teach that certain types of oil-in-water emulsions have antimicrobial, adjuvant, and delivery properties.

A composition of the present invention comprises an effective amount of an effective terpene. An effective (i.e., anti-infective) amount of the terpene is the amount that produces a desired effect, i.e., prevention or treatment of a plant infection. This is the amount that will reach the necessary locations of the plant at a concentration which will kill the infective agent Though less than a full kill can be effective, this will likely have little value to an end user since it is relatively easy to adjust the amount to achieve a full kill. If there were an instance where the amount for a full kill was very close to the phytotoxic amount, an amount that achieves a stable population or stasis of the infective agent can be sufficient to prevent disease progression. An effective (i.e., anti-infective) terpene is one which produces the desired effect, i.e., prevention or treatment of a plant infection, against the particular infective agent(s) with the potential to infect or which have infected the plant(s).

In one embodiment, the most effective terpenes are the C₁₀H₁₆ terpenes. In one embodiment, the more active terpenes for this invention are the ones which contain oxygen. It is preferred for regulatory and safety reasons that food grade terpenes (as defined by the U.S. FDA) be used.

The composition can comprise a single terpene, more than one terpene, a liposome-terpene combination, or combinations thereof. Mixtures of terpenes can produce synergistic effects.

All classifications of natural or synthetic terpenes will work in this invention, e.g., monoterpenes, sesquiterpenes, diterpenes, triterpenes, and tetraterpenes. Examples of terpenes that can be used in the present invention are citral, pinene, nerol, b-ionone, geraniol, carvacrol, eugenol, carvone, terpeniol, anethole, camphor, menthol, limonene, nerolidol, farnesol, phytol, carotene (vitamin A₁), squalene, thymol, tocotrienol, perillyl alcohol, borneol, myrcene, simene, carene, terpenene, and linalool. The list of exempted terpenes found in EPA regulation 40 C. F. R. Part 152 is incorporated herein by reference in its entirety. The terpenes are also known by their extract or essential oil names, such as lemongrass oil (contains citral).

Citral, for example, citral 95, is an oxygenated C₁₀H₁₆ terpene, C₁₀H₁₆O CAS No. 5392-40-53,7-dimethyl-2,6-octadien-1-al.

Plant extracts or essential oils containing terpenes can be used in the embodiments of this invention, as well as the more purified terpenes. Terpenes are readily commercially available or can be produced by various methods known in the art, such as solvent extraction or steam extraction/distillation. Natural or synthetic terpenes are effective in the invention. The method of acquiring the terpene is not critical to the operation of the invention.

The liposome-terpene(s) combination comprises encapsulation of the terpene, attachment of the terpene to a liposome, or is a mixture of liposome and terpene. Alternatively, vehicles other than liposomes can be used, such as microcapsules or microspheres. Since the liposome or encapsulating vehicle serves as a time release device and will not be taken up by the plant, the size and structure of the vehicle can be determined by one of skill in the art based on the desired release amounts and timing. The forms of the compositions that are not taken up by the plant can be used as surface treatments for the plants.

It is known to one of skill in the art how to produce a liposome or other encapsulating vehicle. For example, an oil-in-oil-in-water composition of liposome-terpene can be used.

The composition can further comprise additional ingredients. For example, water (or theoretically, alternatively, any plant-compatible dilutant or carrier), a surfactant, preservative, or stabilizer. However, addition of any additional ingredients will make the composition more difficult for a plant to absorb/take up the composition. Though in theory any plant-compatible dilutant or carrier can be used, any dilutant or carrier other than water would likely not be well accepted by a plant.

Examples of surfactant include polysorbate 20, polysorbate 80, polysorbate 40, polysorbate 60, polyglyceryl ester, polyglyceryl monooleate, decaglyceryl monocaprylate, propylene glycol dicaprilate, triglycerol monostearate, TWEEN, SPAN 20, SPAN 40, SPAN 60, SPAN 80, or mixtures thereof.

The concentration of terpene in the composition is an anti-infective amount. This amount can be from about an infective agent controlling level (e.g., about 20 ppm) to about a phytotoxic level (e.g., about 0.5-1% (5000-10000 ppm) for most plants, though the level is plant specific). This amount can vary depending on the terpene(s) used, the form of terpene (e.g., liposome-terpene), the infective agent targeted, and other parameters that would be apparent to one of skill in the art. One of skill in the art would readily be able to determine an anti-infective amount for a given application based on the general knowledge in the art and guidance provided in the procedures in the Examples given below. A preferred concentration for citral alone being used against Xylella fastidiosa in drench irrigation is 500 ppm.

Concentrations of terpene of about, for example, 20, 30, 40, 50, 60, 70, 80, 90, 100, 110, 125, 130, 140, 150, 175, 200, 225, 250, 300, 350, 400, 450, 500, 600, 750, 800, 1000, 1100, 1250, 1425, 1500, 1750, 2000, 2250, 2500, 3000, 3500, 4000, 4250, 4500, or 4750 ppm can be used as effective concentrations in the compositions and methods of the current invention.

Concentrations of any other ingredients or components can also be readily determined by one of skill in the art using methods known in the art and demonstrated below.

Terpenes have a relatively short life span of approximately 28 days once exposed to oxygen (e.g., air). Testing a plant at 28 days after treatment shows that approximately 99% of the terpene is gone. Terpenes will decompose to CO₂ and water in plants. This decomposition or break down of terpenes in plants is an indication of the safety and environmental friendliness of the compositions and methods of the invention.

The LD₅₀ in rats of citral is approximately 5 g/kg. This also is an indication of the relative safety of these compounds.

A stable suspension of citral can be formed up to about 2500 ppm. Citral can be made into a solution at up to about 500 ppm.

Of the terpenes tested, citral has been found to form a solution at the highest concentration level. Citral will form a solution in water up to about 1000 ppm and is phytotoxic at approximately 5000 ppm.

Various concentrations of citral mixtures were tested against Xylella in vitro and in plants in vivo to determine kill levels for both Xylella and phytotoxicity in the plants (grapes).

TABLE 1
Concentrations of citral vs. effect on Xylella and phytotoxicity
in grape.

| Concentration | Result |
|---|---|
| 62.5 ppm | No complete kill but get stasis |
| 125 ppm | 100% kill |
| 500 ppm | 100% kill |
| >2500 ppm | phytotoxicity |

At sufficiently high levels of terpene, a terpene acts as a solvent and will lyse cell walls.

Approximately 125 ppm is the minimum desired concentration to be used with citral in treatment of Xylella.

If a surfactant is used in the composition, the composition can be effective as a topical application. A composition comprising a terpene, water, and a surfactant forms a suspension of the terpene in the water. It has been observed, as indicated in the Examples below, that plants will not take up a composition which comprises a surfactant. Some terpenes may need a surfactant to form a relatively homogeneous mixture with water.

For internal treatment and/or prevention, a composition comprising a “true” solution of a terpene is desired. A method for making a true solution comprising a terpene is described below.

The composition(s) of the present invention are effective against most infective agents. Examples of infective agents include fungi, viruses, viroids, bacteria, and phytoplasmas/mycoplasmas. Specifically, the composition has been shown to be effective in vitro against bacteria or phytoplasmas. In vivo the composition(s) has been shown to be effective against Xylella fastidiosa or phytoplasmas.

Methods

The invention includes a method of making the composition of the present invention. A method of making a terpene-containing composition that is effective for preventing and/or treating plant infections comprises adding an effective amount of an effective terpene to a carrier.

The terpenes and carriers are discussed above. The concentration at which each component is present is also discussed above. For example, 1000 ppm of citral can be added to water to form a true solution. As another example, 2500 ppm of citral can be added to water with a surfactant to form a stable suspension.

The method can further comprise adding a surfactant to the terepene-containing composition. Concentrations and types of surfactants are discussed above.

The method can further comprise mixing the terpene and carrier (e.g., water). The mixing is under sufficient shear until a “true” solution is formed. Mixing can be done via any of a number of high shear mixers or mixing methods. For example, adding terpene into a line containing water at a static mixer can form a solution of the invention. With the more soluble terpenes, a true solution can be formed by agitating water and terpene by hand (e.g., in a flask). With lesser soluble terpenes, homogenizers or blenders provide sufficient shear to form a true solution. With the least soluble terpenes, methods of adding very high shear are needed or, if enough shear cannot be created, can only be made into the desired mixture by addition of a surfactant and, thus, render these solutions only effective as external surface treatments.

Mixing the terpene and water with a solution-forming amount of shear instead of adding a surfactant will produce a true solution. A plant is capable of taking up a true solution. A solution-forming amount of shear is that amount sufficient to create a true solution as evidenced by a final clear solution as opposed to a cloudy suspension or emulsion.

Citral is not normally miscible in water. Previously in the art a surfactant has always been used to get such a terpene into water. By adding a surfactant, however, plants did not take up such a solution. The surfactant does not go into the plant. Therefore, delivery into the plant has always been a difficulty. The present invention is able to form a solution of up to 1000 ppm, for example, in water by high shear mixing and, thus, overcome this drawback. This solution created by high shear mixing is taken up by plants.

Of the terpenes tested, citral has been found to form a solution at the highest concentration level in water.

The improved results (plant actually takes up solution) with the absence of a surfactant are a result of the terpene forming a “true” solution with water. The presence of the surfactant will only create a suspension of the terpene in the water, which is not taken up.

In a field application, the terpene can be added in line with the water and the high shear mixing can be accomplished by a static inline mixer.

Any type of high shear mixer will work. For example, a static mixer, hand mixer, blender, or homogenizer will work

Infections in or on plants are caused by a variety of organisms. For example, these organisms include bacteria, viruses, mycoplasmas/phytoplasmas, spiroplasmas, or fungi. The present invention is effective against any of these classifications of infective agents, in particular, bacteria, mycoplasmas/phytoplasmas, and spiroplasmas.

One such bacterium is Xylella, such as Xylella fastidiosa. This bacterium inhabits plants' xylem to cause diseases of grapevines, almond, alfalfa, other trees, and crops. Other strains of Xylella cause important diseases of peach, citrus, coffee, and numerous forest tree species.

Plant infections occur in a wide variety of plants. Many of these plants are economically significant crops. Examples of these plants include grapes, stone fruits, coffee, and ornamental trees.

The compositions and methods of the present invention are effective in preventing or treating many, if not all, of these infections in a great variety of plants.

The invention includes a method of treating and/or preventing plant infections. The method comprises administering a composition of the present invention to plants.

The composition of this invention can be administered by a variety of means. For example, the composition can be administered by conventional overhead watering (topical application and/or to be taken up by the plant), drip irrigation, injection, drench or flood irrigation.

As an application in vineyards, the vines can be treated with the composition of the current invention approximately 2 times per year wherein each treatment comprises administration of the composition of the invention twice one week apart.

The life span/breakdown time of the terpenes, as indicated above, should be taken into account when formulating a treatment schedule for prevention and/or treatment according to the present invention.

Terpenes are able to travel up the xylem, cross over to the phloem (such as in the leaves or the stem) and travel down the phloem in order to be able to control spiroplasmas. This appears to be the only way to control spiroplasmas.

### EXAMPLES

The following examples are put forth so as to provide those of ordinary skill in the art with a complete disclosure and description of how the compounds, compositions, articles, devices, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric.

### Example 1

Preparation of a terpene solution as emulsion or suspension using a surfactant

The terpene, terpene mixture, or liposome-terpene(s) combination comprises or consists of a blend of generally recognized as safe (GRAS) terpenes with a GRAS surfactant The volumetric ratio of terpenes is about 1-99%, and the surfactant volumetric ratio is about 1-50% of the solution/mixture. The terpenes, comprised of natural or synthetic terpenes, are added to water. The surfactant is preferably polysorbate 80 or other suitable GRAS surfactant.

### Example 2

### Preparation of a Terpene Solution without Surfactant

Alternatively, the solution can be prepared without a surfactant by placing the terpene, e.g., citral, in water and mixing under solution-forming shear conditions until the terpene is in solution.

0.5 mL citral was added to 1 L water. The citral and water were blended in a household blender for 30 seconds.

Alternatively, moderate agitation also prepared a solution of citral by shaking by hand for approximately 2-3 minutes.

Greater than about zero ppm to about 1000 ppm of natural or synthetic terpenes such as citral, b-ionone, geraniol, carvone, terpeniol, carvacrol, anethole, or other terpenes with similar properties are added to water and subjected to a solution-forming shear blending action that forces the terpene(s) into a true solution. The maximum level of terpene(s) that can be solubilized varies with each terpene. Examples of these levels are as follows.

TABLE 2
Solution levels for various terpenes.

| | |
|---|---|
| Citral | 1000 ppm |
| Terpeniol | 500 ppm |
| b-ionone | 500 ppm |
| Geraniol | 500 ppm |
| Carvone | 500 ppm |

### Example 3

### Potency of Solution

Terpenes will break down in the presence of oxygen.

Citral is an aldehyde and will decay (oxygenate) over a period of days. A 500 ppm solution will lose half its potency in 2-3 weeks.

### Example 4

### Toxicity Trials

Eighteen plants were used to investigate phytotoxic levels of terpene. The periwinkles were grafted with scions from Pierce's disease (PD) Xylella fastidiosa-infected periwinkles. Six Xylella-infected periwinkles were treated with a 1% active terpene mixture. Six plants were treated with 0.5% active terpene mixture. Six were treated with water control.

Two trials were performed. The Trial 1 active mixture was 90% linalool and 10% polysorbate 80. Trial 2 was a repeat of Trial 1 except for the active ingredient (i.e., terpene). The Trial 2 active mixture was 90% citral and 10% polysorbate 80.

Plants were drenched with 500 mL water or treatment on day 1, 14, and 28. Observations were made on day 42.

Three out of six treated at the 1% level died. One out of six at the 0.5% level died. No death was seen in the controls.

Results were the same for each trial. The plants that survived still showed Xylella symptoms.

### Example 5

### In vitro Effectiveness of Terpenes Against Several Microorganisms

In vitro effectiveness of terpene compositions against various organisms was tested. The effectiveness of a terpene mixture solution comprising 10% by volume polysorbate 80, 10% b-ionone, 10% L-carvone, and 70% citral (lemon grass oil) against Escherichia coli, Salmonella typhyimurium, Pasteurella mirabilis, Staphylococcus aureus, Candida albicans, and Aspergillius fumigatus was tested. The terpene mixture solution was prepared by adding terpenes to the surfactant. The terpene/surfactant was then added to water. The total volume was then stirred using a stir bar mixer.

Each organism, except A. fumigatus, was grown overnight at 35-37° C. in tryptose broth A. fumigatus was grown for 48 hours. Each organism was adjusted to approximately 10⁵ organisms/mL with sterile saline. For the broth dilution test, terpene mixture was diluted in sterile tryptose broth to give the following dilutions: 1:500, 1:1000, 1:2000, 1:4000, 1:8000, 1:16,000, 1:32,000, 1:64,000 and 1:128,000. Each dilution was added to sterile tubes in 5 mL amounts. Three replicates of each series of dilutions were used for each test organism. One half mL of the test organism was added to each series and incubated at 35-37° C. for 18-24 hours. After incubation the tubes were observed for growth and plated onto blood agar. The tubes were incubated an additional 24 hours and observed again. The A. fumigatus test series was incubated for 72 hours. The minimum inhibitory concentration for each test organism was determined as the highest dilution that completely inhibits the organism.

TABLE 3
Results of the inhibitory activity of different dilutions of terpene
composition.

| | | | | Growth | | | |
|---|---|---|---|---|---|---|---|
| | Visual Assessment of | | | After Subculture to | | | Mean |
| | Growth* | | | Agar Plates* | | | Inhibitory |
| Organism | 1 | 2 | 3 | 1 | 2 | 3 | Dilution |
| S. typhimurium | 500 | 500 | 500 | 500 | 500 | 500 | 500 |
| E. coli | 1,000 | 1,000 | 1,000 | 1,000 | 1,000 | 1,000 | 1,000 |
| P. mirabilis | 1,000 | 1,000 | 1,000 | 1,000 | 1,000 | 1,000 | 1,000 |
| P. aureginosa | NI** | NI | NI | NI | NI | NI | NI |
| S. aureus | 1,000 | 1,000 | 1,000 | 1,000 | 1,000 | 1,000 | 1,000 |
| C. albicans | 1,000 | 1,000 | 1,000 | 1,000 | 1,000 | 1,000 | 1,000 |
| A. fumigatus | 8,000 | 16,000 | 16,000 | 8,000 | 16,000 | 16,000 | 13,300 |

*The results of the triplicate test with each organism as the reciprocal of the dilution that showed inhibition/killing.
**NI = not inhibited.

### Example 6

### In Vitro Effectiveness of Citral on Xylella Sp

This example shows the bactericidal effect of citral on Xylella sp.

Citral was used undiluted or mixed at a volumetric ratio of 90% citral plus 10% polysorbate 80. Three strains of Xylella were used in this study: Shiraz, Melody, and Coyaga.

The study was as follows:

1. Stock solutions each of citral and citral plus polysorbate 80 were prepared.

2. Stock solutions were diluted in brucella broth 10% (v/v) fetal calf serum to final concentrations of 250, 125, 62.5, and 31.25 ppm. Controls consisted of 10% (v/v) polysorbate 80 in brucella broth, brucella broth alone, and bacteria in brucella broth.

3. A total of 1.0×10⁸ bacteria (0.5 mL) was added to 0.5 mL terpene dilutions (final volume of 1.0 mL) in loosely capped tubes and incubated for 24 hours and 72 hours at 37° C. with continuous mixing. Each citral concentration consisted of three replicates/concentration.

4. Bacterial colony forming units (CFU) were determined visually (i.e., by counting).

The results are summarized in the following table:

TABLE 4
Effect of different citral concentrations on Xylella growth

| | |
|---|---|
| Xylella strain | Terpene concentration (ppm) |

| (10⁸ CFU) | 250 | 125 | 62.5 | 3.25 |
|---|---|---|---|---|
| Shiraz | NG* | NG | TNTC** | TNTC |
| Melody | NG | NG | TNTC | TNTC |
| Coyaga | NG | NG | TNTC | TNTC |

*NG = No Growth
**TNTC = Too Numerous To Count

### Example 7

### Effects of Terpene on Growth of spiroplasmas and Mycoplasma iowae

Effects of neat citral on growth of Spiroplasma citri, S. floricola, S. apis, S. melliferum, and Mycoplasma iowae were studied.

Three concentrations (500 ppm, 250 ppm, and 125 ppm) of citral in sterile DI water were prepared.

Spiroplasmas were grown in R₂ (Chen, T. A., J. M. Wells, and C. H. Liao. 1982. Cultivation in vitro: spiroplasmas, plant mycoplasmas, and other fastidious, walled prokaryotes. pp. 417-446. in Phytopathogenic prokaryotes, V. 2, M. S. Mount and G. H. Lacy (ed.), Academic Press, New York) broth and incubated at 30° C., whereas Mycoplasma iowae were incubated at 37° C. in R₂.

One to 2-day old cultures of each species were observed under a dark-field microscope to ensure cells were in helical form for spiroplasmas and filamentous form for M. iowae before treatment. Cell suspensions were vortexed to ensure they were evenly mixed before and an aliquot of 0.5 mL was dispensed into a sterile tube.

One half of 1 mL of each terpene solution was added into each cell suspension tube. Thus, the final concentrations of citral were 250 ppm, 125 ppm, and 62.5 ppm, respectively. The cell suspension that was added with 0.5 mL of sterile water was used as a control.

The treated cell suspension was incubated for 24 hrs before the color changing units (CCUs) were determined by a 10-fold serial dilution in fresh R₂. All treatments were duplicated. The CCUs were determined to 10⁻⁸ for terpene concentrations of 250 ppm and 125 ppm and to 10⁻⁹ for a terpene concentration of 62.5 ppm and sterile water.

All culture tubes were incubated for 15 days before final reading were taken.

TABLE 5
Results of citral in vitro against spiroplasmas or mycoplasmas.

| | Treatment | | | |
|---|---|---|---|---|
| | Water-treated | 62.5 ppm | 125 ppm | 250 ppm |
| Organism | (CCUs) | | | |
| S. citri | 10⁹ | 10⁹ | 10⁷ | 10⁵ |
| S. melliferum | 10¹⁰ | 10¹⁰ | 10⁸ | 10⁶ |
| S. apis | 10⁹ | 10⁹ | 10⁷ | 10³ |
| S. floricola | 10⁹ | 10⁹ | 10⁶ | 10⁶ |
| M. iowae | 10⁹ | 10⁸ | 10⁸ | 10⁷ |

A comparison was made of the effect of 24-hr. and 48-hr. treatment times. The CCUs were determined by taking treated cell suspension from the same treated tube 24 hrs. or 48 hrs. after treatment.

TABLE 6
24 and 48 hour treatment comparisons.

| | Treatment (ppm) | | | | | | | |
|---|---|---|---|---|---|---|---|---|
| | Water- | Water- | | | | | | |
| | treated | treated | 62.5 | 62.5 | 125 | 125 | 250 | 250 |
| | 24 hr | 48 hr | 24 hr | 48 hr | 24 hr | 48 hr | 24 hr | 48 hr |
| Organism | (CCUs) | | | | | | | |
| S. citri | 10⁹ | 10⁸ | 10⁸ | 10⁷ | 10⁶ | 10⁴ | 10⁴ | 0 |
| S. | 10⁹ | 10⁸ | 10⁹ | 10⁸ | 10⁶ | 10⁵ | 10⁴ | 0 |
| melliferum | | | | | | | | |
| S. apis | ND* | ND | ND | ND | ND | ND | ND | ND |
| S. | ND | ND | ND | ND | ND | ND | ND | ND |
| floricola | | | | | | | | |
| M. iowae | 10⁷ | 10⁶ | 10⁶ | 10⁶ | 10⁷ | 10⁶ | 10⁵ | 10⁴ |

*ND = testing not done

The results indicate that citral could serve as a control for spiroplasmal diseases when used at 250 ppm and treated for 48 hrs.

### Example 8

### Root Uptake Experiments

Various plants were treated to determine whether they would take up various terpene-containing compositions. The plants tested were banana pepper and cherry tomato plants approximately six inches high in two-inch pots with commercial potting soil.

Eight plants were tested. Two were treated with 50 ppm active terpene treatment, two at 250 ppm, two at 500 ppm, and two were water controls. Plants were treated twice per day with 100 mL each treatment Plants were outside in a sunny environment with ideal growing conditions.

Trial 1 active treatment was citral within liposomes, oil-in-oil microencapsulations made with vegetable oil.

Trial 2 active treatment was emulsified citral, 90% citral and 10% polysorbate 80.

After one week, leaf and stem material were taken from the test plants and extracted using isopropyl alcohol. The extract was filtered and shot on a gas chromatograph (GC). No citral was detected in the plant material indicating no uptake with liposomes or surfactant.

### Example 9

### Greenhouse Trial with Phytoplasma

Periwinlde (Catharanthus roseus (L.), white or pink color) was grown under normal greenhouse conditions in one gallon containers with regular potting soil. Periwinkle flowers turn green when aster yellow phytoplasma is present

Each plant was hand-watered with 500 mL of water or terpene composition 500 ppm citral in water was administered to 5 healthy periwinkle plants grafted with scions infected with aster yellow phystoplasma (AYP). The plants were grafted on Day 0. Treatments were applied via water on Day 8 and Day 14 at 500 mL solution per plant Three plants were treated with the terpene solution, and 2 plants were tap water controls.

One of the 2 controls showed typical virescence (green flowers) on Day 64, and symptoms developed over the entire plant. One control remained healthy due to a failed graft. The scion died 4 weeks after grafting and failed to infect the plant.

All three treated plants remained symptomless as of Day 108. Three flowers on one plant showed very light green color on Day 86, but all new flowers remained healthy. This indicates that the three off-color flowers were slightly infected prior to treatment

### Example 10

### Effects of Terpene on Growth of Spiroplasmas and Mycoplasma iowae

Spiroplasmas and Mycoplasma

Spiroplasma citri (R8A2), S. apis (SR-3), S. floricola (23-6), S. melliferum (AS 576), and Mycoplasma iowae (PPAV) were used. All were grown in R₂ broth and incubated at 30° C. except M. iowae at 37° C.

Concentrations of Citral Prepared

Citral was dissolved in sterile water at the following three concentrations: 500, 250, and 125 ppm.

Treatment of Cell Suspensions with Citral

One to two-day old cultures of each strain were vortexed to ensure they were evenly mixed before an aliquot of 0.5 mL was dispensed into a sterile tube. One half of 1 mL of each terpene solution was added into each cell suspension tube. Thus, the final concentrations of citral were 250, 125, and 62.5 ppm, respectively. The cell suspension that was added with 0.5 mL of sterile water was used as control. The treated cell suspension was incubated for 24 hrs. at its respective temperature before the color-changing units (CCUs) were determined by a 10-fold serial dilution in fresh R₂. All treatments were duplicated. The CCUs were determined to 10⁻⁸ for terpene treatments of 250 and 125 ppm and to 10⁻⁹ for terpene treatment of 62.5 ppm and sterile water. All culture tubes were incubated for 15 days before the final readings were taken. An attempt was made to compare the effect of 24-hr. and 48-hr. treatment times for S. citri, S. melliferum, or M. iowae.

Treatment of Aster Yellows Phytoplasma (Ayp)-Grafted Periwinkles with Citral at 500 ppm

Each of five periwinkles was grafted with a scion of AYP-infected periwinkle on Day 0. Three plants were treated with terpene solution, each plant was watered with 500 mL of 500 ppm terpene solution twice on Day 8 and Day 15, respectively. Two plants were treated with tap water (500 mL/plant each time) as controls.

Observation of Symptom Development of the Treated Periwinkles

Treated plants were kept in the greenhouse and fertilized weekly with Peter's 10-10-10 liquid fertilizer. Plants were observed for the development of virescence and phyllody symptoms which are two general symptoms of AYP infection on periwinkles.

Results/Discussion

Treatment of Cell Suspensions with Citral for 24 hr.

The average CCUs for each strain of spiroplasma and mycoplasma treated with various concentrations of citral are shown in Table 7.

TABLE 7
The CCUs for water-treated, 62.5, 125, or 250 ppm
terpene-treated for Spiroplasma citri, S. apis,
S. floricola, S. melliferum, and Mycoplasma iowae.

| | Water- | Citral (ppm) | | |
|---|---|---|---|---|
| | treated | 62.5 | 125 | 250 |
| Spiroplasma citri | 10⁹ | 10⁹ | 10⁷ | 10⁵ |
| S. melliferum | 10¹⁰ | 10¹⁰ | 10⁸ | 10⁶ |
| S. apis | 10⁹ | 10⁹ | 10⁷ | 10³ |
| S. floricola | 10⁹ | 10⁹ | 10⁶ | 10⁶ |
| Mycoplasma iowae | 10⁹ | 10⁸ | 10⁸ | 10⁷ |

There was an obvious decrease of spiroplasma cells when terpene was used at 125 and 250 ppm.

Comparison of 24-hr. and 48-hr. Treatment of Cell Suspensions with Citral

The average CCUs for each strain of spiroplasma and mycoplasma treated with various concentrations of citral for 24-hr. or 48-hr. are shown in Table-8.

TABLE 8
The CCUs for water-treated, 62.5, 125, or 250 ppm terpene-
treated for 24-hr. or 48-hr. for Spiroplasma citri, S. melliferum,
and Mycoplasma iowae.

| Water- | Citral (ppm) | | |
|---|---|---|---|
| treated | 62.5 | 125 | 250 |

| | | | | | |
|---|---|---|---|---|---|
| Spiroplasma citri | 24 hr | 10⁹ | 10⁸ | 10⁶ | 10⁴ |
| | 48 hr | 10⁸ | 10⁷ | 10⁴ | 0 |
| S. melliferum | 24 hr | 10⁹ | 10⁹ | 10⁶ | 10⁴ |
| | 48 hr | 10⁸ | 10⁸ | 10⁵ | 0 |
| Mycoplasma iowae | 24 hr | 10⁷ | 10⁶ | 10⁷ | 10⁵ |
| | 48 hr | 10⁶ | 10⁶ | 10⁶ | 10⁴ |

There was an obvious decrease of spiroplasma cells when treatment was increased to 48-hr. When 250 ppm terpene was used, no cells of S. citri or S. melliferum survived the 48-hr. treatment. It was, however, not enough to kill M. iowae.

Symptom Development of AYP-Grafted Periwinkles that were Treated

All three citral treated periwinkles remained symptomless as of Day 174, whereas one of the water-treated control periwinkles began to show virescent (greenish) flowers on Day 64. This control plant continued to develop more virescent flowers and phyllody. One control periwinkle remained symptomless. The scion of this control plant died 22 days after grafting which may have been a result of an unsuccessful transmission, hence remained asymptomatic. One of the three terpene treated periwinkles developed two light green flowers in one branch on Day 86 for a period of two to three weeks. This seemed to indicate that the treatment delayed the symptom development for 22 days. However, there have not been any more light green or green flowers developed since then. On the contrary, the plant has remained symptomless to date. It was unclear how the two light green flowers developed. The first treatment started 8 days after the initial grafting. Whether 8 days was enough for the AYP to cause the slight change in petal color merits further investigation. It was obvious that the terpene was able to suppress the symptom development induced by AYP or other phytoplasmal diseases. The inhibitory effect of terpene on M iowae was not as strong as it was on spiroplasmas, which warrants further investigation.

### Example 11

### Minimum Inhibitory Concentrations (MICs) of Terpene on Growth of Xylella fastidiosa Strains

Xylella fastidiosa Strains used

Five grape strains (Cayuga, Melody, Shiraz, 3SV, and Yugo), 2 sycamore strains (SLS-DC and SLS#61) and 1 strain each of peach (4#5), plum (2#6), pecan (4BD2), and oleander (#6) were used. All were grown in PW agar and incubated at 30° C. Culture plates were sub-cultured on a weekly basis.

Preparation of Terpene Solutions

Citral was dissolved in sterile water at 500, 250, and 125 ppm concentrations.

Treatment of Cell Suspension with Terpene

Cell suspension of each strain were prepared by re-suspending cells scraped from a 7-day old agar culture plate into 3 mL of fresh PW broth. Cell suspensions of each strain were vortexed to ensure even mixing before an aliquot of 0.5 mL was dispensed into a sterile tube. One of half of 1 mL of each terpene solution was added into each cell suspension tube. Thus, the final concentrations of terpene were 250, 125, and 62.5 ppm, respectively. The cell suspension that was added with 0.5 mL of sterile water was used as control. The treated cell suspension was incubated for 24 hrs. at 30° C. before the color-changing units (CCUs) were determined by a 10-fold serial dilution in fresh PW broth. All treatments were duplicated. The CCUs were determined to 10⁻⁹ for all treatments. All culture tubes were incubated for 20 days before the final readings were taken. The MIC was the lowest concentration at which no cell survived the treatment

Treatment of X. fastidiosa-Infected Grapevines

A total of 21 grapevines showing Pierce's disease symptoms were selected for treatment. They were 3-year old vines from Montmorenci Vineyard in Aiken, S. C. Fifteen vines were treated with terpene, while 6 vines were treated with water as controls. Each vine was drenched with 2 L of 500 ppm terpene near the trunk area, whereas each control vine was drenched with 2 L water. Two treatments were performed for each vine, the first treatment on Day 0 and the second on Day 7.

Isolation of X. fastidiosa from Petioles of Terpene-Treated and Control Grapevines

Three to four leaves with petioles from each vine were randomly picked on Day 7 right before the second treatment and were shipped to the lab in a cooler with ice. Samples were used for the isolation of the bacterium in PW agar plates on Day 8. The same number of leaves with petioles were collected on Day 22 and were used for isolations on Day 23. One gram of petioles from each vine was surface-sterilized with 20% CLOROX for 15 min. followed by 3 rinses in sterile water (3 min. per rinse). The sterilized petioles were minced in 3 mL of PW broth. The sap was streaked onto PW agar with an inoculation loop. The PW agar plates were then placed in a plastic bag and incubated at 30° C. for the colony development for up to 4 wks. Colony observation was done using a dissecting scope weekly.

Growth Measurements of Terpene-Treated and Control Grapevines

Growth comparison between terpene-treated and control vines were conducted by measuring the two longest branches and two shortest branches of each vine on Day 206. The average length of the four branches of each vine was compared.

Results/Discussion

Minimal Inhibitory Concentrations (MICs) of each X. fastidiosa Strain

Based on the color-changing units from the 10-fold serial dilutions, it was concluded that terpene at 250 ppm killed cells of all 11 strains of X. fastidiosa after 24-hr. treatment. The MICs, defined as the lowest concentrations in which no cells survived the treatment, were 125 ppm for 4 grape strains, 2 sycamore strains, and 1 peach strain, and 62.5 ppm for strains from grape, plum, pecan, and oleander.

TABLE 9
The MICs of citral for 11 strains of X. fastidiosa.

| Strain | Disease incited | MIC (ppm) |
|---|---|---|
| Cayuga | Pierce's disease of grapevine | 125 |
| Melody | Pierce's disease of grapevine | 125 |
| Shiraz | Pierce's disease of grapevine | 125 |
| 3SV | Pierce's disease of grapevine | 125 |
| Yugo | Pierce's disease of grapevine | 62.5 |
| SLS-DC | Sycamore leaf scorch | 125 |
| SLS#61 | Sycamore leaf scorch | 125 |
| 4#5 | Phony disease of peach | 125 |
| 2#6 | Plum leaf scald | 62.5 |
| 4BD2 | Pecan leaf scorch | 62.5 |
| #6 | Oleander leaf scorch | 62.5 |

Isolations of Xylella fastidiosa from Petioles of Terpene-Treated and Control Grapevines

Of the samples collected one week after the first treatment, 4 out of 6 (67%) control vines had typical X. fastidiosa colonies, whereas only 4 out of 15 (27%) of treated vines had X. fastidiosa colonies. Of those collected on Day 21, two weeks after the second treatment, the same 67% of control vines gave positive isolation of X fastidiosa, whereas only 3 out of 15 (20%) treated vines gave positive isolation of X fastidiosa. Based on both results, it was clear that terpene killed the bacteria in 11 or 12 out of 15 vines, or 6 or 7 out of 10 vines assuming that only 67% of 15 treated vines were actually diseased vines.

Growth Measurements of Terpene-Treated and Control Grapevines

Four branches (two longest and two shortest) from each of the 21 vines were measured on Day 206 for the growth comparison between terpene treated and control vines. The average lengths of the measured branches for each vine are shown in Table 10.

TABLE 10
Average of measured branches.

| Row and | | Length of branch (in.) | | | | Average. branch |
|---|---|---|---|---|---|---|
| vine # | Terpene/water | 1 | 2 | 3 | 4 | length (in.) |
| R19V108 | Terpene | 52 | 54 | 24 | 30 | 40 |
| R19V106 | Terpene | 27 | 43 | 52 | 25 | 37 |
| R19V105 | Terpene | 22 | 36 | 33 | 24 | 29 |
| R19V104 | Terpene | 15 | 17 | 16 | 9 | 14 |
| R19V103 | Terpene | 38 | 30 | 25 | 26 | 30 |
| R18V108 | Terpene | 28 | 17 | 9 | 14 | 17 |
| R18V107 | Terpene | 54 | 41 | 25 | 15 | 34 |
| R18V106 | Terpene | 33 | 27 | 16 | 12 | 22 |
| R18V105 | Terpene | 29 | 28 | 18 | 16 | 23 |
| R18V104 | Terpene | 31 | 24 | 17 | 16 | 22 |
| R18V103 | Terpene | 29 | 28 | 15 | 9 | 20 |
| R17V107 | Terpene | 32 | 29 | 13 | 19 | 23 |
| R17V106 | Terpene | 18 | 16 | 11 | 12 | 14 |
| R17V105 | Terpene | 15 | 37 | 32 | 18 | 26 |
| R17V103 | Terpene | 9 | 11 | 8 | 7 | 9 |
| Average | Terpene | — | — | — | — | 24 |
| R19V101 | Water | 15 | 17 | 13 | 17 | 16 |
| R19V100 | Water | 14 | 28 | 24 | 13 | 20 |
| R18V101 | Water | 33 | 27 | 16 | 12 | 22 |
| R18V100 | Water | 23 | 22 | 13 | 13 | 18 |
| R17V100 | Water | 17 | 31 | 38 | 11 | 24 |
| R17V99 | Water | 6 | 12 | 12 | 4 | 9 |
| Average | Water | — | — | — | — | 18 |

Based on the average branch length, the treated vines seemed to grow 6 inches longer than control vines. One of the treated vines (R19V108) showed more vigorous growth as compared to the water-treated control vine (R19V101). Their growth and yield of grapes will be compared at the end of the season.

Day 252 Bacteria Isolation and ELISA Testing

The above treated vines were sampled on Day 252 for isolation and ELISA tests on the bacteria. Three out of 15 terpene-treated vines showed positive results presence of bacteria, whereas three out of six non-treated control vines gave positive results. This result was similar to that which was obtained from samples that were collected and assayed in Month 1 and Month 2, indicating that the treatment was effective up to Day 252.

Other vines at the Montmorenci Vineyard were treated the following year. Those vines were first treated on Day 206 and Day 213 and were sampled on Day 252 for isolation and ELISA tests of the bacteria. Four out of 15 terpene-treated vines show positive results, while 5 out of five non-treated control vines gave positive detection of the bacteria.

### Example 12

### Phytoplasma Treatment

A total of 12 healthy vines were treated with 4 L each of water control, 500 ppm citral, 1000 ppm citral, and 2500 ppm citral. Weekly observations for 3 weeks afterward showed no phytoplasma on any plants, indicating a minimum 5-fold safety margin.

Note: the 2500 ppm level was a suspension rather than a solution and would not have been taken up by the roots but rather have coated the root hairs.

### Example 13

### Increased Fruit Yield

The plants used in Example 11 were followed for about 1 year. The treated grapevines yielded an average of about 4.8 lbs of fruit per vine. The untreated controls yielded about 4.5 lbs of fruit per vine. This shows an average increased yield of about 6.25%.

The yield is expected to increase more in following years.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

Throughout this application, various publications are referenced. The disclosures of these publications in their entireties are incorporated by reference into this application in order to more fully describe the state of the art to which this invention pertains.

1. A composition for treating and/or preventing disease by an infectious agent in plants comprising an effective amount of at least one effective terpene.

2. The composition of claim 1 wherein the composition is a solution capable of being taken up by a plant.

3. The composition of claim 2 wherein the solution capable of being taken up by a plant is a true solution.

4. The composition of claim 1 further comprising water.

5. The composition of claim 1 further comprising a surfactant and water.

6. The composition of claim 5 wherein the surfactant is polysorbate 20, polysorbate 80, polysorbate 40, polysorbate 60, polyglyceryl ester, polyglyceryl monooleate, decaglyceryl monocaprylate, propylene glycol dicaprilate, triglycerol monostearate, TWEEN, SPAN 20, SPAN 40, SPAN 60, SPAN 80, or mixtures thereof.

7. The composition of claim 1 further comprising a stabilizer.

8. The composition of claim 1 wherein the at least one terpene is a mixture of different terpenes.

9. The composition of claim 1 wherein the at least one terpene is a terpene-liposome combination.

10. The composition of claim 1 wherein the terpene comprises citral, pinene, nerol, b-ionone, geraniol, carvacrol, eugenol, carvone, terpeniol, anethole, camphor, menthol, limonene, nerolidol, farnesol, phytol, carotene (vitamin A₁), squalene, thymol, tocotrienol, perillyl alcohol, borneol, myrcene, simene, carene, terpenene, linalool, or mixtures thereof.

11. The composition of claim 1 wherein the terpene is citral, geraniol, thymol, or linalool.

12. The composition of claim 1 wherein composition comprises about 1 to 99% by volume terpenes and about 1 to 99% by volume surfactant.

13. The composition of claim 1 wherein the terpene comprises between about 20 ppm and about 5000 ppm.

14. The composition of claim 1 wherein the terpene comprises about 125 ppm.

15. The composition of claim 1 wherein the terpene comprises about 250 ppm.

16. The composition of claim 1 wherein the terpene comprises about 500 ppm.

17. The composition of claim 1 wherein the terpene is citral and the effective amount is 500 ppm.

18. The composition of claim 1 wherein the terpene is effective against bacteria, mycoplasmas/phytoplasmas, and/or fungi.

19. The composition of claim 1 wherein the terpene is effective against bacteria.

20. The composition of claim 1 wherein the terpene is effective against phytoplasmas.

21. A composition for treating and/or preventing disease by an infectious agent in plants comprising a true solution comprising an effective amount of at least one effective terpene and water.

22. A method for treating and/or preventing disease by an infectious agent in a plant comprising administering a composition comprising an effective amount of an effective terpene to the plant.

23. The method of claim 22 wherein the composition further comprises water.

24. The method of claim 22 wherein the composition further comprises a surfactant.

25. The method of claim 22 wherein the administration is by spraying or watering the plants with the composition.

26. The method of claim 22 wherein the administration is by injecting plants with the composition.

27. The method of claim 26 wherein the injection comprises injecting the composition into the xylem of the plant.

28. The method of claim 22 further comprising making a composition comprising an effective amount of an effective terpene.

29. The method of claim 22 wherein the plants are grape vines, stone fruit trees, coffee, or ornamental plants.

30. The method of claim 22 wherein the plants are grapevines.

31. The method of claim 22 wherein the plants are infected with an infective agent.

32. The method of claim 31 wherein the infective agent is bacteria, mycoplasmas/phytoplasmas, and/or fungi.

33. The method of claim 31 wherein the infective agent is bacteria.

34. The method of claim 31 wherein infective agent is phytoplasma.

35. The method of claim 28 wherein the making a composition comprises mixing an effective amount of an effective terpene and water.

36. The method of claim 35 wherein the mixing is done at a solution-forming shear until formation of a true solution of the terpene and water.

37. The method of claim 36 wherein the terpene mixed is into a true solution in water without a surfactant by high shear or high pressure blending or agitation.

38. The method of claim 36 wherein the solution-forming shear mixing is via a static mixer.

39. A method for treating and/or preventing disease by an infectious agent in a plant comprising administering a composition comprising an effective amount of an effective terpene and water to the plant.

40. The method of claim 39 wherein the terpene is citral.

41. The method of claim 39 wherein the composition is a true solution.

42. A method for making a terpene-containing composition effective for treating and/or preventing disease by an infectious agent in a plant comprising mixing a composition comprising a terpene and water at a solution-forming shear until a true solution of the terpene is formed.

43. A method for making a terpene-containing composition capable of plant root uptake and effective for treating and/or preventing disease by an infectious agent in a plant comprising adding terpene to water, and mixing the terpene and water under solution-forming shear conditions until a true solution of terpene and water forms.

44. A method for making the composition of claim 1 comprising mixing a terpene with a carrier.

45. A method for using the composition of claim 1 comprising administering the composition of claim 1 to infected plants.
